(19) United States
(12) Patent Application Publication
(10) Pub. No.: US 2007/0173472 A1
(43) Pub. Date: Jul. 26, 2007

(54) METHODS AND REAGENTS FOR THE TREATMENT OF APOPTOSIS-RELATED DISORDERS

(75) Inventors: Jeff MacKeigan, Swampscott, MA (US); Leon Murphy, Allston, MA (US); Peter Finan, Marblehead, MA (US); Ellen Triantafellow, Brighton, MA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 11/466,517

(22) Filed: Nov. 20, 2006

Related U.S. Application Data
(60) Provisional application No. 60/711,533, filed on Aug. 26, 2005.

(51) Int. Cl. A61K 48/00; C12Q 1/68

(52) U.S. Cl. 514/44; 435/6

(57) ABSTRACT

The present invention relates to novel uses and reagents relating to apoptosis-related disorders. In particular, modulation of PI3K class IIα activity is shown to influence apoptosis, and PI3K class IIα is thought to be cellular survival factors. Based on these findings the present invention provides therapeutic methods and pharmaceutical compositions useful for the treatment of apoptosis-related disorders. In addition, PI3K class IIα is provided as a target for the development of therapeutics. Accordingly, the invention provides screening methods for identifying candidate compounds that modulate PI3K class IIα activity and may therefore be used to treat apoptosis-related disorders such as cardiovascular disorders, autoimmune disorders, neurodegenerative disorders, and cancer.

### BACKGROUND OF THE INVENTION

Apoptosis, or “programmed cell death,” is a normal feature of an organism's differentiation and maturation, through which its cells die via a specific suicide process. Cells “commit suicide” when they outlive their purpose, become defective, or age, and apoptosis prevents cells from, among other things, accumulating and forming tumors.

Apoptosis is morphologically distinctive from necrosis, and is associated with normal physiology. (Kerr et al. (1972) Br J Cancer 26(24): 239). Growing cells feature a precisely controlled number of divisions, which slow during the aging process, ultimately ending with apoptosis. Programmed cell death is also a critical function of cell-related immunity. One way in which the body wards against foreign invaders is via the ability of cytotoxic T lymphocytes (CTLs) to induce target cells to kill themselves via apoptosis. When CTLs cells kill other cells, they activate the latent apoptosis pathway in their targets.

Aberrant, unregulated, and/or disease-related apoptosis is complicit in a variety of medical conditions, and is the desired target of certain treatments. For instance, cancer is often marked by too little apoptosis, whereas unchecked apoptosis can lead to neurodegenerative disorders. Mutations in the p53 gene are very often found in cancer cells, preventing necessary apoptosis and helping cancer cells skirt death, and resulting in cellular proliferation gone awry. Defects that lead to slowing or cessation of the apoptotic machinery are also associated with autoimmune diseases such as lupus erythematosus and rheumatoid arthritis.

“Survival factors,” such as growth factors and interleukins, prolong cell survival by inhibiting apoptosis, important for the maintenance of normal tissue homoeostasis and response to infection or injury. When the survival factor is removed, the default apoptotic death program is triggered. By way of example, mechanisms that control the accumulation of neutrophils at sites of inflammation most likely limit the synthesis of neutrophil survival factors in inflammatory and structural cells. (Simon, HU (2003) Eur Respir J Suppl. 44:20s).

Phosphoinositide 3-kinases (hereafter, “PI3Ks”) are enzymes that phosphorylate the 3-hydroxyl position of the inositol ring of phosphoinositides (“PIs”), and are involved in diverse cellular events such as cell migration, cell proliferation, oncogenic transformation, cell survival, and intracellular trafficking of proteins. There are three classes of PI3Ks, with a variety of isoforms and types within; most of the PI3K biological activity has been described exclusively about Class I PI3Ks. Elucidation of the other PI3K classes is of importance to the scientific community, particularly in light of evidence suggesting that class II PI3Ks (e.g., PI3K class IIα) may play a role as survival factors.

### SUMMARY OF THE INVENTION

The present invention relates to modulating PI3K class IIα for the treatment, amelioration, and diagnosis of apoptosis-related disorders in a patient, such as disorders associated with aberrant or dysregulated apoptosis; for example, disorders associated with too much apoptosis (marked by concomitant cell loss), or too little (marked by cell accumulation), as compared to normal physiological conditions. Said apoptosis-related disorders include but are not limited to cancer, autoimmune disorders, cardiovascular disorders, and neurodegenerative disorders.

In a first aspect the invention provides methods for the treatment of diseases related to aberrant apoptosis comprising administering an effective amount of an agent inhibiting the expression of the gene encoding PI3K class IIα or inhibiting an activity of a PI3K class IIα gene product. In one embodiment, these methods of treatment are used in disorders characterized by a shortage of apoptosis, e.g., in cancer and autoimmune disorders.

In one aspect of the invention, the agent is an inhibitory nucleic acid capable of specifically inhibiting PI3K class IIα, preferably an antisense oligonucleotide compound, more preferably an siRNA compound.

In another aspect the invention provides a method for the treatment of diseases related to aberrant apoptosis comprising administering an effective amount of an agent enhancing the expression of the gene encoding PI3K class IIα or agonizing an activity of a PI3K class IIα gene product. In a preferred embodiment, these methods of treatment are used in disorders characterized by an overabundance of apoptosis, e.g., in neurodegenerative and cardiovascular disorders.

In another aspect of the invention, compositions of matter are included that are capable of modulating PI3K class IIα. In a preferred embodiment, said compositions of matter include nucleic acids capable of specifically modulating PI3K class IIα, preferably an antisense oligonucleotide compound, more preferably an siRNA compound.

In yet another aspect, the present invention relates to pharmaceutical compositions comprising an effective amount of an agent modulating (e.g., inhibiting or enhancing) the expression of the gene encoding PI3K class IIα or modulating (e.g., inhibiting or enhancing) the activity of PI3K class IIα gene product. Preferably, the pharmaceutical compositions comprises an effective amount of an antisense oligonucleotide which is capable of modulating PI3K class IIα.

In a further aspect, the present invention relates to methods for identifying compounds useful for treatment of apoptosis-related disorders comprising: (a) contacting a PI3K class IIα polypeptide with a test compound; and (b) detecting modulation of PI3K class IIα biological activity. One way to test modulation of a PI3K class IIα biological activity is to assay the activity of downstream targets of PI3K class IIα (e.g., of mTOR, a downstream target of the PI3K/Akt pathway).

In yet another aspect, the present invention relates to methods for determining whether a patient is suffering from or at risk for an apoptosis-related disorder, comprising: (a) providing a test biological sample obtained from the subject; and (b) determining whether the level of expression of PI3K class IIα nucleic acid or polypeptide in the biological sample differs from the PI3K class IIα level of expression in a comparable biological sample obtained from a healthy subject. A difference in said levels of expression is an indication that the test subject is suffering from or is at risk for an apoptosis-related disorder.

In yet another aspect, the present invention relates to a method for modulating the amount or activity of one or more polypeptides regulated by PI3K class IIα comprising modulating the expression of PI3K class IIα (e.g., via administration of inhibitory nucleic acids). The PI3K class IIα regulated genes or proteins may be selected from the group consisting of IκB, Bad, caspase 9, forkhead-related transcription factor 1, and mammalian target of rapamycin (mTOR).

Other features and advantages of the invention will be apparent from the following detailed description, the drawings, and the claims.

### BRIEF DESCRIPTION OF THE FIGURES

FIG. 1 shows a comparison between PI3K class IIα RNA levels in Hela cells transfected with different siRNAs generated against PI3K class IIα, as measured by quantitative RT-PCR. Lane one shows a control, and the next four lanes reflect different siRNAs directed against PI3K class IIα, as described herein.

FIGS. 2a and 2b show a Western blot analysis of PI3K class IIα knockdown with antibodies against PI3K class IIα protein. Apoptosis was measured by cleavage of caspase 9 and PARP after 72 hours of siRNA against PI3K class IIα.

FIG. 3 shows a comparison of apoptosis occurring in Hela cells as a result of transfection with different siRNAs directed against PI3K class IIα, as measured by DNA fragmentation ELISA.

FIGS. 4a and 4b show plasma membrane blebbing and cell viability visualized by phase contrast microscopy in Hela and U20S cells, respectively.

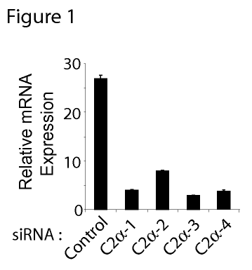

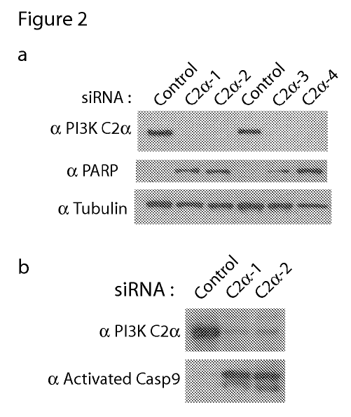

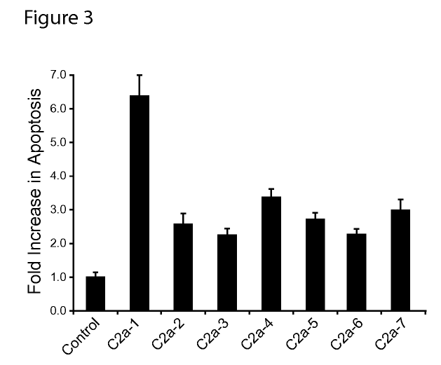

### DETAILED DESCRIPTION OF THE INVENTION

It is contemplated that the invention described herein is not limited to the particular methodology, protocols, and reagents described, as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Harlow, E. and Lane, eds., 1988, “Antibodies: A Laboratory Manual”, Cold Spring Harbor Press, Cold Spring Harbor, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

In the present description, the term “treatment” includes both prophylactic or preventive treatment as well as curative or disease suppressive treatment, including treatment of patients at risk of apoptotic-related disorders as well as ill patients. This term further includes the treatment for the delay of progression of the disease.

By “suppress and/or reverse,” e.g., a disorder associated with apoptosis in a patient (e.g., an autoimmune disease), Applicants mean to abrogate said condition, or to render said condition less severe than before or without the treatment.

As used herein, “modulate” indicates the ability to control or influence directly or indirectly, and by way of non-limiting examples, can alternatively mean inhibit or stimulate, agonize or antagonize, hinder or promote, and strengthen or weaken.

“Cure” as used herein means to lead to the remission of the disorder associated with apoptosis in a patient, or of ongoing episodes thereof, through treatment.

The terms “prophylaxis” or “prevention” mean impeding the onset or recurrence of apoptosis-related disorders, e.g., autoimmune disorders.

“Delay of progression” as used herein means that the administration of an agent or pharmaceutical composition to patients in a pre-stage or in an early phase of a disorder (e.g., a associated with aberrant apoptosis in a patient (e.g., an autoimmune disorders)) prevents the disease from evolving further, or slows down the evolution of the disease in comparison to the evolution of the disease without administration of the pharmaceutical composition.

“Apoptosis-related disorders” include but are not limited to cancers, cardiovascular disorders, neurodegenerative disorders, and autoimmune disorders.

“Cardiovascular disorders” include but are not limited to stroke, acute coronary syndromes including unstable angina, thrombosis and myocardial infarction; atherosclerosis (or arteriosclerosis); plaque rupture; both primary and secondary (in-stent) restenosis in coronary or peripheral arteries; transplantation-induced sclerosis; peripheral limb disease; ischemic heart disease (e.g., angina pectoris, myocardial infarction, and chronic ischemic heart disease); hypertensive heart disease; pulmonary heart disease; valvular heart disease (e.g., rheumatic fever and rheumatic heart disease, endocarditis, mitral valve prolapse, and aortic valve stenosis); preeclampsia; peripheral vascular disease; atrial or ventricular septal defect; myocardial disease (e.g., myocarditis, myocardial ischemia, congestive cardiomyopathy, and hypertrophic cariomyopathy); and diabetic complications (including ischemic heart disease, peripheral artery disease, congestive heart failure, retinopathy, neuropathy and nephropathy).

“Neurodegenerative disorders” include but are not limited to Huntington's disease, Parkinson's Disease, Alzheimer's Disease, dystonia, dementia, multiple sclerosis, Amyotrophic Lateral Sclerosis (ALS), and Creutzfeld-Jacob Disease.

“Autoimmune disorders” include but are not limited to rheumatoid arthritis, Graves' disease, multiple sclerosis, scleroderma, autoimmune hepatitis, fibromyalgia, myasthenia gravis (MG), systemic lupus erythematosis (SLE), graft rejection (e.g., allograft rejection), and T cell disorders (including acquired immune deficiency syndrome (AIDS)).

As used herein, the term “cancer” includes solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc. Both human and veterinary uses are contemplated.

As used herein the terms “normal mammalian cell” and “normal animal cell” are defined as cells that are growing under normal growth control mechanisms (e.g., genetic control) and display normal cellular differentiation. Cancer cells differ from normal cells in their growth patterns and in the nature of their cell surfaces. For example cancer cells tend to grow continuously and chaotically, without regard for their neighbors, among other characteristics well known in the art.

As used herein, the term “inhibitory nucleic acid” refers to nucleic acid compounds capable of producing gene-specific inhibition of gene expression. Typical inhibitory nucleic acids include, but are not limited to, antisense oligonucleotides, triple helix DNA, RNA aptamers, ribozymes and short inhibitory RNAs (“siRNAs”). For example, knowledge of a nucleotide sequence may be used to design siRNA or antisense molecules which potently inhibit the expression of class II PI3Ks. Similarly, ribozymes can be synthesized to recognize specific nucleotide sequences of a gene and cleave it. Techniques for the design of such molecules for use in targeted inhibition of gene expression are well known to one of skill in the art.

The invention relates to the methods and compositions for treatment, diagnosis, and/or amelioration of apoptosis-related disorders, e.g., autoimmune disorders. In a preferred embodiment, the invention relates to the use of inhibitory nucleic acids to treat apoptosis-related disorders, e.g., autoimmune disorders.

Phosphoinositide 3-kinases, referred to herein as PI3Ks, are enzymes that activate intracellular signaling molecules upon growth factors binding their cell surface receptors. These lipid kinases phosphorylate the inositol ring of phosphatidylinositol and related compounds at the 3-prime position; they are capable of phosphorylating both phosphatidylinositols (also referred to as “PtdIns”) and phosphoinositides (“PIs”, which are phosphorylated versions of phosphatidylinositols).

The products of these reactions serve as secondary messengers in growth signaling pathways, influencing such cellular events as cell survival, migration, motility, and proliferation; oncogenic transformation; tissue neovascularization; and intracellular protein trafficking. Cell-surface receptors induce the production of second messengers such as phosphatidylinositol 4,5-bisphosphate 3, which convey signals from the cell surface to cytoplasm. These secondary messengers activate the PI3K-dependent protein kinase-1, which in turn activates the kinase Akt. Akt activation leads to phosphorylation of proteins leading to cell survival. (Cantley et al. (1999) PNAS 96:4240).

By way of example, Akt phosphorylates IκB, thereby activating NFκB and promoting cellular survival. Akt also phosphorylates Bad (a proapoptotic Bcl-2 family member) and caspase 9, in both cases blocking the induction of apoptosis. Other downstream targets of Akt include forkhead-related transcription factor 1 and mammalian target of rapamycin (mTOR).

PI3Ks are grouped in three classes, categorized according to their structure, substrate specificity, physiological function, and tissue-specificity. (Domin et al. (1997) FEBS Lett. 410:91). Class I PI3Ks are mostly cytosolic, are heterodimers comprising a p110 catalytic subunit and an adaptor/regulatory subunit, and are further divided into two classes: Class IA PI3Ks consist of a p110 catalytic subunit that associates with an SH2 domain-containing subunit p85, and is activated by the majority of tyrosine kinase-coupled transmembrane receptors; class IB PI3K consists of a p101 regulatory subunit that associates with p110γ catalytic subunit, and is activated by heterotrimeric G-protein-coupled-receptors. (Katso et al. (2001) Annu. Rev. Cell Dev. Biol. 17:615). Class II PI3Ks consist of three isoforms, as discussed herein. Class III PI3Ks utilize only phosphatidylinositol as a substrate, and play an essential role in protein trafficking through the lysosome. (Volinia, et al. (1995) EMBO J. 14:3339).

Class II PI3Ks are predominantly associated with membrane fractions of cells, are characterized by a C2 domain at their C-terminus, and consist of three isoforms (PI3K-C2α, PI3K-C2β, and PI3K-C2γ). (Sheikh, et al. (2003) BMC Clin. Pathol. 3:1). Expression of PI3K-C2α and PI3K-C2β is ubiquitous, whereas PI3K-C2γ is mainly found in the liver. The C2 domain of PI3K class IIs can bind in vitro to phospholipids in a calcium-independent manner (Arcaro et al. (1998) J. Biol. Chem. 273:33082). Deletion of this domain does not affect the subcellular localization of this class of PI3Ks. Contrarily, less is known about the large N-terminal region of PI3K class IIs, and homology to known proteins has not been demonstrated.

PI3K class IIα is ubiquitously expressed, and is generally known to be activated downstream of growth factors insulin, epidermal growth factor (EGF), monocyte chemotactic peptide-1 (MCP-1), and protein-derived growth factor (PDGF). When these growth factors-known to promote cell proliferation-bind their cell-surface receptors, PI3K class IIα are activated and transmit intracellular secondary signals.

As shown herein, modulating PI3K class IIα influences apoptosis. The Examples section of the present application (infra) describes in detail how inhibiting the expression of PI3K class IIα results in cellular changes indicative of apoptosis, strongly suggesting PI3K class IIα function as cell survival factors.

Apoptosis is morphologically distinctive from necrosis, and is associated with normal physiology. (Kerr et al. (1972) Br J Cancer 26(24): 239). During development and later, excess numbers of neurons, lymphocytes and many other kinds of cells die through this genetically programmed sequence of changes; for instance, human fingers are separated during development because embryonic cells that joined them were programmed to undergo cell death. Growing cells feature a precisely controlled number of divisions, which slow during the aging process, ultimately ending with apoptosis.

Programmed cell death is also a critical function of cell-related immunity. One way in which the body wards against foreign invaders is via the ability of cytotoxic T lymphocytes (CTLs) to induce target cells to kill themselves via apoptosis. Mature CTLs secrete toxic molecules to its targets, such as cells infected with malignant viruses, to trigger apoptosis therein. When CTLs cells kill other cells, they activate the latent apoptosis pathway in their targets.

Apoptosis can also be induced—for example, by DNA damage that exceeds the capacity of repair mechanisms, in order to rid the bodies of cellular deficiencies. Cells respond to DNA damage by increasing their production of p53, a tumor suppresser and potent inducer of apoptosis. Apoptosis is used in the thymus to eliminate self-reactive T lymphocytes, thereby avoiding autoimmunity once CTLs have completed their duties. The chromatin-associated enzyme poly (ADP-ribose) polymerase (“PARP”) is a chromatin-associated enzyme thought to induce apoptosis.

Aberrant, unregulated, and/or disease-related apoptosis is complicit in a variety of conditions, and is the desired target of certain treatments. For instance, cancer is often marked by too little apoptosis, whereas unchecked apoptosis can lead to neurodegenerative disorders. Mutations in the p53 gene are very often found in cancer cells, preventing necessary apoptosis and helping cancer cells skirt death, and resulting in cellular proliferation gone awry. Defects that lead to slowing or cessation of the apoptotic machinery are also associated with autoimmune diseases such as lupus erythematosus and rheumatoid arthritis.

Unlike necrotic cell death, in which cells swell and burst, spilling their contents over neighboring ones (often triggering an inflammatory response), with apoptosis, the cell nucleus condenses (nuclear chromatin condensation), and the cell itself shrivels as the cytoplasm shrinks. Apoptosis is also characterized by dilated endoplasmic reticulum and membrane blebbing; mitochondria remain unchanged morphologically.

The changes in the apoptotic cell trigger phagocytosis by non-activated macrophages, which engulf and degrade cellular corpses. Macrophages appear to recognize apoptotic cells via several different recognition systems. For example, there is good evidence that apoptotic cells lose the normal phospholipid asymmetry in their plasma membrane, as manifested by the exposure of normally inward-facing phosphatidyl serine on the external face of the bilayer. Macrophages can recognize this exposed lipid headgroup via an unknown receptor, triggering phagocytosis.

Another biochemical hallmark of apoptotic death which increasingly appears is the activation of caspases, which are cysteine proteases related to ced-3, or the “death gene” of the nematode Caenorhabditis elegans. Caspases seem to be widely-expressed in an inactive proenzyme form in most cells. Their proteolytic activity is characterized by their unusual ability to cleave proteins at aspartic acid residues, although different caspases have different fine specificities involving recognition of neighboring amino acids. Active caspases can often activate other pro-caspases, allowing initiation of a protease cascade. Persuasive evidence that these proteases are involved in most examples of apoptotic cell death has come from the ability of specific caspase inhibitors to block cell death, as well as the demonstration that knockout mice lacking caspase 3, 8 and 9 fail to complete normal embryonic development.

“Survival factors,” such as growth factors and interleukins, prolong cell survival by inhibiting apoptosis, important for the maintenance of normal tissue homoeostasis and the response to infection or injury. When the survival factor is removed, the default apoptotic death program is triggered. By way of example, mechanisms that control the accumulation of neutrophils at sites of inflammation most likely limit the synthesis of neutrophil survival factors in inflammatory and structural cells. (Simon, HU (2003) Eur Respir J Suppl. 44:20s).

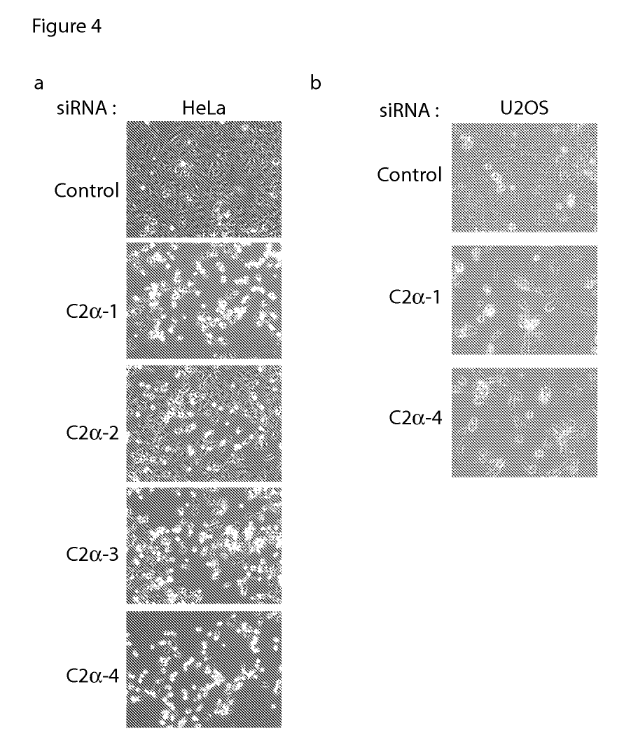

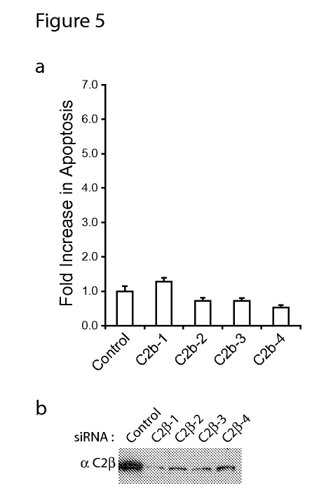

When PI3K class IIα nucleotide or protein expression is inhibited, such as by the siRNAs listed herein, apoptosis is allowed to occur unimpeded. For this reason, the experiments depicted in the present application describe symptoms of apoptosis that were triggered upon the inhibition of PI3K class IIα, such as plasma membrane blebbing (e.g., in FIGS. 4a and 4b). The experiments also reveal caspase 9 activation and PARP cleavage as a consequence of inhibiting PI3K class IIα.

Caspases are a family of proteins that are one of the main effectors of apoptosis. These cysteine proteases exist within the cell as inactive pro-forms or zymogens. These zymogens can be cleaved to form active enzymes following the induction of apoptosis, whereby they are capable of cleaving one another and other proteins at the C-terminus of aspartic acid residues.

Induction of apoptosis results in the activation of an initiator caspase, such as caspases 8, 9, or 10, which then activate other caspases in a proteolytic cascade leading to the activation of effector caspases. The effector caspases (e.g., caspases 3 and 6) are responsible for the cleavage of the key cellular proteins that leads to the typical changes observed in cells undergoing apoptosis, such as digestion of structural proteins in the cytoplasm, degradation of chromosomal DNA, and phagocytosis of the cell.

PARP, or poly(ADP-ribose) polymerase, is a 116 kDa protein involved in the repair of DNA, in differentiation, and in chromatin structure formation. PARP activation and subsequent cleavage (e.g., by caspases) have active and complex roles in apoptosis. During apoptosis, this protein is cleaved by caspases into an 89 kDa fragment and a 24 kDa fragment, detection of a which is a hallmark of apoptosis. In contrast to measuring active caspase 3, which is degraded during apoptosis via the ubiquitin/proteosome pathway, measuring PARP cleavage allows sustained signal detection even in late stages of apoptosis.

Since PI3K class IIs are thought to be survival factors, as shown in the experiments described herein, when PI3K class II nucleotide or protein expression is suppressed, apoptosis occurs unimpeded. Dysregulation of apoptosis in the form of uninterrupted cell death is associated with a variety of conditions, including AIDS, neurodegenerative disorders, blood diseases (such as aplastic anemia), or cardiovascular disorders (e.g., low-oxygen injuries such as heart attacks or stroke.

By way of example, the loss of cardiac monocytes through apoptosis contributes to the progression of heart failure. Studies have shown that cardiac myocyte apoptosis also occurs after acute myocardial infarction, as well as in the hypertrophied heart and the aging heart, conditions frequently associated with the development of heart failure. (Sabbah et al. (1998) Prog Cardiovasc Dis. 40(6):549).

By way of further example, neurodegenerative disorders are associated with an overabundance of apoptosis and resultant cell loss. ALS is characterized by spinal motor neurons apoptosis, leading to paralysis and death; Alzheimer's disease and Parkinson's disease are also associated with aberrant neuronal loss. Said apoptosis under these conditions can arise from a variety of triggers, including a lack of neurotrophic support, overactivation of glutamate receptors and calcium influx, and increased oxidative stress. (Mattson (2000) Nat Rev Mol Cell Biol. 1(2):120).

An appropriate treatment regime for the above-listed disorders, therefore, is artificial inhibition of apoptosis, such as via the enhancement of PI3K class IIα. One aspect of the present invention is a method for the treatment of diseases related to aberrant apoptosis comprising administering an effective amount of an agent that enhances the expression of the genes encoding PI3K class IIα, or that enhances an activity of a PI3K class IIα gene product.

The present invention also provides for a pharmaceutical composition comprising an effective amount of an agent enhancing the expression of the gene encoding PI3K class IIα or enhancing an activity of PI3K class IIα gene product.

Since PI3K class IIα is thought to be a survival factor, as shown in the experiments described herein, when PI3K class IIα nucleotide or protein expression is suppressed, apoptosis occurs unimpeded. Dysregulation of apoptosis in the form of a shortage of programmed cell death (and a concomitant overabundance of cells), which can be alleviated by inhibiting PI3K class IIα, is associated with a variety of conditions, including cancers, autoimmune diseases, and viral infections.

By way of example, cancers cells grow in an unmitigated fashion in the body because of defects in apoptosis. Cancerous cells avoid apoptosis through a variety of mechanisms, such as by overexpressing certain proteins and drowning out apoptosis-induction signals as a result (e.g., in the case of some B-cell leukemias and lymphomas, which express high levels of Bcl-2 (a family of key regulators of apoptosis)); and by secreting “decoy” molecules that bind to one of a ligand-receptor binding pair necessary for apoptosis, thereby preventing the necessary binding event (e.g., in the case of lung and colon cancers, which prevent FasL from binding Fas). Cancer-causing viruses are able to protein proteins that bind and inactivate apoptosis promoters (e.g., p53).

By way of further examples, autoimmune disorders occur when the body fails to clear itself of immune cells once cell-mediated immune responses wane. In normal physiology, apoptosis would be induced in said immune cells, removing them from a healthy system; instead, due to defects in apoptotic mechanisms, immune cells attack the body's own constituents rather than foreign invaders.

An appropriate treatment regime for the above-listed disorders, therefore, is artificial enhancement of apoptosis, such as via the inhibition of PI3K class IIα. One aspect of the present invention is a method for the treatment of diseases related to aberrant apoptosis comprising administering an effective amount of an agent that inhibits the expression of the gene encoding PI3K class IIα, or inhibiting an activity of a PI3K class IIα gene product.

In one aspect of the invention, the agent is an inhibitory nucleic acid capable of specifically inhibiting PI3K class IIα. Typical inhibitory nucleic acids include, but are not limited to, antisense oligonucleotides, triple helix DNA, RNA aptamers, ribozymes and short inhibitory RNAs (“siRNAs”). In a preferred embodiment of the present invention, the agent is an antisense oligonucleotide compound, more preferably an siRNA compound

In an especially preferred embodiment of the present invention, the siRNA compounds are selected from the following: agaggaagtgctgcagaataa (known as C2a-1; SEQ ID NO:1); ttgaagagagatcgacagcaa (known as C2a-2; SEQ ID NO:2); aaggatttcagctaccagtta (known as C2a-3; SEQ ID NO:3); cacaaggaagcttacctatct (known as C2a-4; SEQ ID NO:4); ttagcttctttactgattctg (known as C2a-5; SEQ ID NO:5); ttgaatacttgtaagttctgg (known as C2a-6; SEQ ID NO:6); and cagaatcagtaaagaagctaa (known as C2a-7; SEQ ID NO:7).

The present invention also provides for a pharmaceutical composition comprising an effective amount of an agent inhibiting the expression of the gene encoding PI3K class IIα or inhibiting the an activity of PI3K class IIα gene product. Preferably, the PI3K class IIα inhibitor is an antisense oligonucleotide or an siRNA. More preferably, the PI3K class IIα inhibitor is an siRNA compound selected from the following: agaggaagtgctgcagaataa (known as C2a-1; SEQ ID NO:1); ttgaagagagatcgacagcaa (known as C2a-2; SEQ ID NO:2); aaggatttcagctaccagtta (known as C2a-3; SEQ ID NO:3); cacaaggaagcttacctatct (known as C2a-4; SEQ ID NO:4); ttagcttctttactgattctg (known as C2a-5; SEQ ID NO:5); ttgaatacttgtaagttctgg (known as C2a-6; SEQ ID NO:6); and cagaatcagtaaagaagctaa (known as C2a-7; SEQ ID NO:7).

The present invention further provides for methods for identifying compounds useful for treatment of apoptosis-related disorders comprising: (a) contacting a PI3K class II polypeptide with a test compound; and (b) detecting modulation of PI3K class II biological activity. One way to test modulation of a PI3K class IIα biological activity is to assay the presence or activity of downstream targets of PI3K class IIα.

In yet another aspect, the present invention relates to methods for determining whether a patient is suffering from or at risk for an apoptosis-related disorder, comprising: (a) providing a test biological sample obtained from the subject; and (b) determining whether the level of expression of PI3K class IIα nucleic acid or polypeptide in the biological sample differs from the PI3K class IIα level of expression in a comparable biological sample obtained from a healthy subject. A difference in said levels of expressions is an indication that the test subject is suffering from or is at risk for an apoptosis-related disorder.

In yet another aspect, the present invention relates to a method for modulating the activity of one or more polypeptides regulated by PI3K class IIα comprising modulating the expression of PI3K class IIα (e.g., via administration of inhibitory nucleic acids). The PI3K class IIα regulated genes or proteins may be selected from the group consisting of IκB, Bad, caspase 9, forkhead-related transcription factor 1, and mammalian target of rapamycin (mTOR).

RNAi

Inhibitory nucleic acid compounds of the present invention may be synthesized by conventional means on a commercially available automated DNA synthesizer, e.g. an Applied Biosystems (Foster City, Calif.) model 380B, 392 or 394 DNA/RNA synthesizer, or like instrument. Phosphoramidite chemistry may be employed. The inhibitory nucleic acid compounds of the present invention may also be modified, for instance, nuclease resistant backbones such as e.g., phosphorothioate, phosphorodithioate, phosphoramidate, or the like, described in many references may be used. The length of the inhibitory nucleic acid has to be sufficient to ensure that the biological activity is inhibited. Thus, for instance in case of antisense oligonucleotides, has to be sufficiently large to ensure that specific binding will take place only at the desired target polynucleotide and not at other fortuitous sites. The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, and the like. Preferably, the antisense oligonucleotides of the invention have lengths in the range of about 15 to 40 nucleotides. More preferably, the oligonucleotide moieties have lengths in the range of about 18 to 25 nucleotides.

Double-stranded RNA, i.e., sense-antisense RNA, also termed small interfering RNA (siRNA) molecules, can also be used to inhibit the expression of nucleic acids for PI3K class IIs. RNA interference is a method in which exogenous, short RNA duplexes are administered where one strand corresponds to the coding region of the target mRNA (Elbashir et al. (2001) Nature 411:494). Upon entry into cells, siRNA molecules cause not only degradation of the exogenous RNA duplexes, but also of single-stranded RNAs having identical sequences, including endogenous messenger RNAs. Accordingly, siRNA may be more potent and effective than traditional antisense RNA methodologies since the technique is believed to act through a catalytic mechanism. Preferred siRNA molecules are typically from 19 to 25 nucleotides long, preferably about 21 nucleotides in length and comprise the sequence of a nucleic acid for E2-EPF5. Effective strategies for delivering siRNA to target cells include, for example, transduction using physical or chemical transfection. Alternatively siRNAs may be expressed in cells using, e.g., various PolIII promoter expression cassettes that allow transcription of functional siRNA or precursors thereof. See, for example, Scherr et al. (2003) Curr. Med. Chem. 10(3):245; Turki et al. (2002) Hum. Gene Ther. 13(18):2197; Cornell et al. (2003) Nat. Struct. Biol. 10(2):91. The invention also covers other small RNAs capable of mediating RNA interference (RNAi) such as for instance micro-RNA (miRNA) and short hairpin RNA (shRNA).

Screening Assays

In a particularly preferred embodiment, PI3K class IIs are provided as targets for the screening for therapeutics useful in the treatment of diseases in which aberrant apoptosis plays a role (e.g., autoimmune, cardiovascular, cancer-related, or neurodegenerative disorders). The present invention provides methods for identifying a compound useful for modulating PI3K class IIα comprising (a) contacting a PI3K class IIα polypeptide with a test compound; and (b) detecting a modulation of PI3K class IIα biological activity. The modulation is usually detected with respect to a control reaction lacking the test compound. Modulation as used herein refers to an increase or reduction of the biological activity, preferably by at least 10%, at least 20%, at least 30%, at least 50%, or at least 100%.

In another embodiment, the present invention provides methods for identifying compounds useful for treatment of a disease associated with aberrant apoptosis, comprising: (a) contacting a PI3K class IIα polypeptide with a test compound; and (b) detecting modulation of PI3K class IIα biological activity. One way to test modulation of a PI3K class IIα biological activity is to assay the presence or activity of downstream targets of PI3K class IIα.

Compound screening assays may include cell-based or cell-free systems. Cell-based systems can be native, i.e., cells that normally express the PI3K class IIα, as a biopsy or expanded in cell culture. In one embodiment, however, cell-based assays involve recombinant host cells expressing PI3K class IIα. Determining the ability of test compounds to interact with the PI3K class IIα can also comprise determining the ability of test compounds to preferentially bind to the polypeptide as compared to the ability of a known binding molecule to bind to the polypeptide.

In yet another embodiment, an assay of the present invention is a cell-free assay in which a protein or biologically active portion thereof is contacted with a test compound and the ability of the test compound to bind to PI3K class IIα proteins or biologically active portion thereof is determined. Binding of the test compound to PI3K class IIα proteins can be determined either directly or indirectly as described above. In a preferred embodiment, the assay includes contacting the PI3K class IIα proteins or biologically active portion thereof with compounds known to bind PI3K class IIα proteins to form an assay mixture, contacting the assay mixture with a test compound, and determining the ability of the test compound to interact with PI3K class IIα proteins, wherein determining the ability of the test compound to interact with PI3K class IIα proteins comprises determining the ability of the test compound to preferentially bind to PI3K class IIα proteins or biologically active portions thereof as compared to the known compound.

The polypeptides can be used to identify compounds that modulate PI3K class IIα activity. Such compounds, for example, can increase or decrease affinity for PI3K class IIα protein substrate, such as phosphatidylinositols (also referred to as “PtdIns”) or phosphoinositides (“PIs”, which are phosphorylated versions of phosphatidylinositols). Such compounds could also, for example, increase or decrease the rate of binding to these substrates, could compete with these substrates for binding to the PI3K class IIα, or could displace these substrates bound to PI3K class IIα.

PI3K class IIα, derivatives and fragments can be used in fast screening methods e.g. automated high-throughput screens (HTS) to assay candidate compounds for the ability to bind to the PI3K class IIα. Numerous suitable fast screening assays are known to the skilled person.

These compounds can be further screened against functional PI3K class IIα to determine the effect of the compound on PI3K class IIα. Compounds can be identified that activate (agonist) or inactivate (antagonist) PI3K class IIα to a desired degree. Modulatory methods can be performed in vitro (e.g., by culturing the cell with the agent) or, alternatively, in vivo (e.g., by administering the agent to a subject). PI3K class IIα polypeptides can be used to screen a compound for the ability to stimulate or inhibit interaction between the PI3K class IIα protein and target molecules that normally interact with the PI3K class IIα protein. The target can be any component of the pathway with which PI3K class IIα protein normally interacts. The assay includes the steps of combining the PI3K class IIα protein with a candidate compound under conditions that allow the PI3K class IIα protein or fragments to interact with target molecules, and to detect the formation of a complex between the PI3K class IIα protein and the targets, or to detect the biochemical consequence of the interaction with PI3K class IIα and the targets. Any of the associated effects of PI3K class IIα functions can be assayed. This includes the production of phosphorylated phosphatidylinositols or phosphoinositides, modulation of downstream targets of PI3K class IIα, or cessation or enhancement of apoptosis.

Determining the ability of PI3K class IIα to bind to target molecules can also be accomplished using a technology such as real-time Bimolecular Interaction Analysis (BIA). As used herein, “BIA” is a technology for studying biospecific interactions in real time, without labeling any of the interactants (e.g., BIAcore®). Changes in the optical phenomenon surface plasmon resonance (SPR) can be used as an indication of real-time reactions between biological molecules. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145). Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 97:6378-6382); (Felici (1991) J. Mol. Biol. 222:301-310).

Candidate compounds include, for example, (1) peptides such as soluble peptides, including Ig-tailed fusion peptides and members of random peptide libraries (see, e.g., Lam et al. (1991) Nature 354:82-84; Houghten et al. (1991) Nature 354:84-86) and combinatorial chemistry-derived molecular libraries made of D- and/or L-configuration amino acids; (2) phosphopeptides (e.g., members of random and partially degenerate, directed phosphopeptide libraries, see, e.g., Songyang et al. (1993) Cell 72:767-778); (3) antibodies (e.g., polyclonal, monoclonal, humanized, anti-idiotypic, chimeric, and single chain antibodies as well as Fab, F(ab′)2, Fab expression library fragments, and epitope-binding fragments of antibodies); and (4) small organic and inorganic molecules (e.g., molecules obtained from combinatorial and natural product libraries).

In one aspect, compounds identified by the screening methods in accordance with the present invention are provided. Such compounds are preferably low molecular weight compounds or antibodies, in particular monoclonal antibodies, or inhibitory nucleic acids. The compounds preferably have a modulatory effect on apoptosis, i.e. they inhibit or promote apoptosis, the determination of which may be made by methods known in the art. Such methods include for instance detection of cellular morphological changes (e.g., membrane blebbing, nuclear condensation), detection of PARP cleavage and/or caspase activation, and detection of phosphatidylinositols (PtdIns) or phosphoinositides (PIs) phosphorylation.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

In more than one embodiment of the above assay methods of the present invention, it may be desirable to immobilize PI3K class IIα to facilitate separation of complexed from uncomplexed forms of the protein, as well as to accommodate automation of the assay. Binding of a test compound to PI3K class IIα protein, or to a protein target, can be accomplished in any vessel suitable for containing the reactants. Examples of such vessels include microtitre plates, test tubes, and microcentrifuge tubes. In one embodiment, a fusion protein can be provided which adds a domain that allows the protein to be bound to a matrix. For example, glutathione-S-transferase/kinase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and the non-adsorbed PI3K class IIα protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding determined using standard techniques.

Other techniques for immobilizing proteins on matrices can also be used in the screening assays of the invention. For example, PI3K class IIα protein can be immobilized utilizing conjugation of biotin and streptavidin. Biotinylated PI3K class IIα protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with PI3K class IIα protein or target molecules can be derivatized to the wells of the plate, and unbound PI3K class IIα protein trapped in the wells by antibody conjugation. Methods for detecting such complexes, in addition to those described above for the GST-immobilized complexes, include immunodetection of complexes using antibodies reactive with the PI3K class IIα protein or target molecules.

In yet another aspect of the invention, the PI3K class IIα protein can be used as a “bait protein” in a two-hybrid assay or three-hybrid assay (see, e.g., U.S. Pat. No. 5,283,317; Zervos et al., 1993 Cell 72:223-232; Madura et al., 1993 J. Biol. Chem. 268:12046-12054; Bartel et al., (1993) Biotechniques 14:920-924; Iwabuchi et al., 1993 Oncogene 8:1693-1696; and Brent WO94/10300), to identify other proteins which bind to the PI3K class IIα protein. Such PI3K class IIα-binding proteins are also likely to be involved in the propagation of signals by the PI3K class IIα protein.

The two-hybrid system is based on the modular nature of most transcription factors, which consist of separable DNA-binding and activation domains. Briefly, the assay utilizes two different DNA constructs. In one construct, the gene that codes for a PI3K class IIα protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a kinase dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the PI3K class IIα protein which interacts with the protein.

This invention further pertains to novel agents identified by the above-described screening assays. Accordingly, it is within the scope of this invention to further use an agent identified as described herein in an appropriate animal model. For example, an agent identified as described herein (e.g., a PI3K class IIα modulating agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Pharmaceutical Compositions

An additional aspect of the invention relates to the administration of a pharmaceutical composition, in conjunction with a pharmaceutically acceptable carrier, for any of the therapeutic effects discussed above. Such pharmaceutical compositions comprise an effective amount of an agent modulating the expression of the gene encoding PI3K class IIα or modulating an activity of a PI3K class IIα gene product.

They may for instance comprise antibodies, mimetics, agonists, antagonists, or inhibitory nucleic acids of PI3K class IIα in accordance with the present invention. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, fragments thereof, antibodies, agonists, antagonists or inhibitors of PI3K class IIα which ameliorates the symptoms or conditions of disorders relating to aberrant apoptosis. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

### EXAMPLES

### Example 1

### Generation of siRNA Sequences

The siRNA sequence design uses a BIOPREDsi potency predictor algorithm to score 21-mer oligoribonucleotides. Top scoring sequences are examined for theoretical selectivity against a specified transcriptome, according to experimentally defined selectivity criteria siRNAs. In addition, siRNAs were synthesized by Qiagen (Qiagen, Valencia, Calif.) and Dharmacon (Lafayette, Colo.) as 21-nt oligoribonucleotides with a 19 base pair duplex region and two deoxynucleotide overhangs on the 3′-terminus of each strand. The DNA of the sense strand was a dTdT, whereas the overhang of the antisense strand was complementary to the target mRNA.

The siRNA sequences generated are as follows:

| | | SEQ |
|---|---|---|
| siRNA | | ID |
| Name | Sequence | NO: |
| C2a-1 | Agaggaagtgctgcagaataa | 1 |
| C2a-2 | Ttgaagagagatcgacagcaa | 2 |
| C2a-3 | Aaggatttcagctaccagtta | 3 |
| C2a-4 | Cacaaggaagcttacctatct | 4 |
| C2a-5 | Ttagcttctttactgattctg | 5 |
| C2a-6 | Ttgaatacttgtaagttctgg | 6 |
| C2a-7 | Cagaatcagtaaagaagctaa | 7 |

### Example 2

### Apoptosis ELISA

siRNAs were transfected using Oligofectamine (Invitrogen, Carlsbad, Calif.) into HeLa cells seeded 24 hours earlier in 96-well plates (3000 cells/well). In each siRNA experiment, negative control scrambled siRNAs were used as siRNA controls. After 72 hours of target knockdown, quantification of apoptotic cell death was determined by an ELISA that measures cytoplasmic histone-DNA fragments produced during apoptosis (Roche, Indianapolis, Ind.). Next, the 96-well plates were centrifuged (200×g) for 10 minutes, supernatant discarded, and lysis buffer added. Following lysis, the samples were centrifuged and 20 μl of the supernatant transferred to a strepavidin-coated microtiter plate. Anti-histone biotin and anti-DNA peroxidase antibodies were added to each well, and the plate incubated at room temperature for 2 hours. After three washes with buffer, the peroxidase substrate was added to each well. Following five minute incubation, the plates were read at 405 nm in a microplate reader. The enrichment of histone-DNA fragments is expressed as fold-increase in absorbance as compared to control (nonsilencing) siRNAs. Pro-apoptotic siRNAs were identified by a ≧2-fold increase in apoptosis over scrambled siRNA control.

### Example 3

### Cell Lysis and Immunoblotting

Cell extracts were prepared by collecting and washing cells in ice-cold PBS and harvesting in lysis buffer (pH 7.2; 10 mM KPO₄, 1 mM EDTA, 10 mM MgCl₂, 50 mM β-glycerophosphate, 5 mM EGTA, 0.5% NP-40, 0.1% Brij-35, 1 mM sodium orthovanadate, 40 μg phenylmethylsulfonyl fluoride/ml, 10 μg leupeptin/ml, 5 μg pepstatin A/ml). Extracts were centrifuged at 15,000 r.p.m. for 10 min at 4° C. and cell lysates immunoblotted using anti-PI3K p170, anti-PI3K ClassII beta (BD Biosciences, San Jose, Calif.), anti-cleaved-caspase 9, anti-PARP (Cell Signaling, Beverly, Mass.) and tubulin (Sigma, St. Louis, Mo.).

### Example 4

### Total RNA Isolation and Assay by Quantitative Real Time PCR (Q-PCR)

Total RNA was extracted 30 hours after transfection from HeLa cells and purified using RNeasy kit (Qiagen,). Primer pairs and FAM-labelled TaqMan probes for real time PCR were designed against PI3K isoforms from TaqMan Gene Expression Assays (Applied Biosystems, Foster City, Calif.). For the Q-PCR reaction, 8 ng cDNA was mixed with 5′ and 3′ primers (0.9 μM each), and TaqMan probe (0.25 μM) in a total volume of 20 μl following the TaqMan Universal PCR reagent kit protocol (Applied Biosystems). Real time PCR was performed in a PRISM 7900HT detection system as follows: 2 minutes at 50° C., 10 minutes denaturation at 95° C. followed by 40 cycles of denaturation for 15 sec at 95° C. and annealing and elongation for 1 min at 60° C. Comparative C_{T} method used for relative expression curves on an ABI PRISM 7900HT detection system (Applied Biosystems, Foster City, Calif.).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

We claim:

1. A method for the treatment of an apoptosis-related disorder comprising administering an effective amount of an agent modulating the expression of the gene encoding a PI3K class IIα or modulating an activity of a PI3K class IIα gene product.

2. A method according to claim 1 wherein the apoptosis-related disorder is a cardiovascular disorder.

3. A method according to claim 1 wherein the apoptosis-related disorder is a neurodegenerative disorder.

4. A method according to claim 1 wherein the apoptosis-related disorder is a cancer.

5. A method according to claim 1 wherein the apoptosis-related disorder is an autoimmune disorder.

6. A method according to claim 1 wherein the agent is an inhibitory nucleic acid.

7. A method according to claim 6 wherein the agent is an siRNA selected from the group consisting of SEQ ID NOs:1-7.

8. A pharmaceutical composition comprising an effective amount of an agent capable of modulating the expression of the gene encoding PI3K class IIα or modulating the activity of a PI3K class IIα gene product and pharmaceutically acceptable carrier.

9. A pharmaceutical composition according to claim 8 wherein the PI3K class IIα modulator is an inhibitory nucleic acid.

10. A pharmaceutical composition according to claim 9 wherein PI3K class IIα inhibitor is an siRNA selected from the group consisting of SEQ ID NOs:1-7.

11. A method for identifying a compound useful for treatment of apoptosis-related disorders comprising:
(a) contacting a PI3K class IIα polypeptide a test compound; and
(b) detecting modulation of PI3K class IIα biological activity.

12. The method of claim 11, wherein the apoptosis-related disorder is selected from a cardiovascular disorder, a neurodegenerative disorder, an autoimmune disorder, and cancer.

13. A method of determining whether a patient is suffering from or at risk for an apoptosis-related disorder, comprising:
a) providing a test biological sample obtained from a subject; and
b) determining whether the level of expression of a PI3K class IIα nucleic acid or polypeptide in the biological sample differs from the PI3K class IIα level of expression in a comparable biological sample obtained from a healthy subject.

14. The method of claim 13, wherein the apoptosis-related disorder is selected from a cardiovascular disorder, a neurodegenerative disorder, an autoimmune disorder, and cancer.


SEQUENCE LISTING (7 sequences)

SEQ ID NO: 1 - DNA, 21 bp, Artificial Sequence
  Synthesized siRNA sequence
agaggaagtgctgcagaataa

SEQ ID NO: 2 - DNA, 21 bp, Artificial Sequence
  Synthesized siRNA sequence
ttgaagagagatcgacagcaa

SEQ ID NO: 3 - DNA, 21 bp, Artificial Sequence
  Synthesized siRNA sequence
aaggatttcagctaccagtta

SEQ ID NO: 4 - DNA, 21 bp, Artificial Sequence
  Synthesized siRNA sequence
cacaaggaagcttacctatct

SEQ ID NO: 5 - DNA, 21 bp, Artificial Sequence
  Synthesized siRNA sequence
ttagcttctttactgattctg

SEQ ID NO: 6 - DNA, 21 bp, Artificial Sequence
  Synthesized siRNA sequence
ttgaatacttgtaagttctgg

SEQ ID NO: 7 - DNA, 21 bp, Artificial Sequence
  Synthesized siRNA sequence
cagaatcagtaaagaagctaa